(19) United States
(12) Patent Application Publication
(10) Pub. No.: US 2026/0259290 A1
(43) Pub. Date: Sep. 3, 2026

(54) SYSTEM AND METHOD FOR OPTIMAL MAGNETIC RESONANCE EXAM WITH RETROSPECTIVE REFORMATTING WORKFLOW THAT LEVERAGES INTEGRATED GUIDELINES

(71) Applicant: GE Precision Healthcare LLC, Waukesha, WI (US)

(72) Inventors: Tisha Anie Abraham, Bangalore (IN); Dattesh Dayanand Shanbhag, Bangalore (IN); Sajith Rajamani, Bangalore (IN); Chitresh Bhushan, Schenectady, NY (US); Uday Damodar Patil, Bangalore (IN)

(21) Appl. No.: 19/068,382

(22) Filed: Mar. 3, 2025

(51) Int. Cl. G01R 33/54; A61B 5/00; A61B 5/055; G16H 10/60; G16H 30/40

(52) U.S. Cl. CPC G01R 33/543; A61B 5/055; A61B 5/7264; G16H 10/60; G16H 30/40

(57) ABSTRACT

A method includes receiving at a protocol optimizer an electronic medical record for the subject, wherein the electronic medical record includes a scan order for scanning a region of interest of the subject for a patient specific clinical indication with a magnetic resonance scanner. The method also includes utilizing the protocol optimizer to automatically access one or more initial protocols specific to the patient specific clinical indication from radiology guidelines and/or a protocol library. The method further includes generating with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

### BACKGROUND

The subject matter disclosed herein relates to medical imaging and, more particularly, to a system and a method for optimal magnetic resonance (MR) exam with retrospective reformatting workflow that leverages integrated guidelines.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During magnetic resonance imaging (MRI), when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Certain protocols for scanning a region of interest of a patient require taking multiple scans two-dimensional (2D) scans of the region of interest for different contrasts. These scan protocols can be lengthy increasing the discomfort of the patient and hinder the scan throughput at the imaging site. Further, these scan protocols may not be adjusted to be specific to the individual patient.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for performing a scan of a subject utilizing a magnetic resonance imaging (MRI) system is provided. The computer-implemented method includes receiving, via a processing system including one or more processors, at a protocol optimizer an electronic medical record for the subject, wherein the electronic medical record includes a scan order for scanning a region of interest of the subject for a patient specific clinical indication with a magnetic resonance scanner. The computer-implemented method also includes utilizing, via the processing system, the protocol optimizer to automatically access one or more initial protocols specific to the patient specific clinical indication from radiology guidelines and/or a protocol library. The computer-implemented method further includes generating, via the processing system, with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

In another embodiment, a system for performing a scan of a subject utilizing an MRI system is provided. The system includes a memory encoding processor-executable routines. The system also includes a processing system including one or more processors and configured to access the memory and to execute the processor-executable routines, wherein the routines, when executed by the processing system, cause the processing system to perform actions. The actions include receiving at a protocol optimizer an electronic medical record for the subject, wherein the electronic medical record includes a scan order for scanning a region of interest of the subject for a patient specific clinical indication with a magnetic resonance scanner. The actions also include utilizing the protocol optimizer to automatically access one or more initial protocols specific to the patient specific clinical indication from radiology guidelines and/or a protocol library. The actions further include generating with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

In a further embodiment, a non-transitory computer-readable medium, the computer-readable medium including processor-executable code that when executed by a processor, causes the processor to perform actions. The actions include receiving at a protocol optimizer an electronic medical record for a subject, wherein the electronic medical record includes a scan order for scanning a region of interest of the subject for a patient specific clinical indication with a magnetic resonance scanner. The actions also include utilizing the protocol optimizer to automatically access one or more initial protocols specific to the patient specific clinical indication from radiology guidelines and/or a protocol library. The actions further include generating with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique;

FIG. 2 illustrates a flow diagram of a method for performing a scan of a patient utilizing the MRI system in FIG. 1, in accordance with aspects of the present disclosure;

FIG. 3 illustrates a schematic diagram of a workflow for performing a scan of a patient, in accordance with aspects of the present disclosure;

FIG. 4 depicts an example recommendation provided on a graphical user interface of a display for a temporal lobe epilepsy protocol, in accordance with aspects of the present disclosure;

FIG. 5 depicts an example recommendation provided on a graphical user interface of a display for a complete pituitary protocol, in accordance with aspects of the present disclosure;

FIG. 6 depicts a sagittal image and a coronal image acquired with two 2D acquisitions for an axial slice prescription;

FIG. 7 depicts a thoraco-lumbar spine sagittal T2 image and a thoraco-lumbar spine coronal T2 image derived from a single 3D acquisition utilizing the method in FIG. 2 and the workflow in FIG. 3, in accordance with aspects of the present disclosure;

FIG. 8 depicts an image of a brain of a subject derived from a 2D acquisition and a reformatted image of the brain from a 3D acquisition, in accordance with aspects of the present disclosure;

FIG. 9 depicts an image of a brain of a subject derived from a 2D acquisition and images of the brain from a 3D acquisitions (and associated reformatted images), in accordance with aspects of the present disclosure;

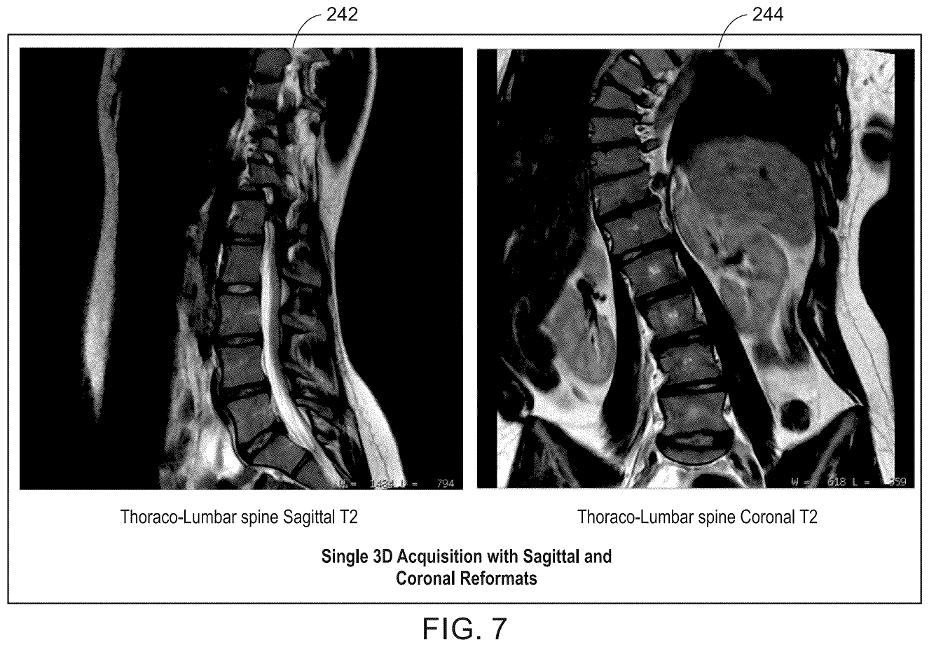

FIG. 7 is an MR image obtained of an anatomic landmark of interest (e.g., brain) of a subject;

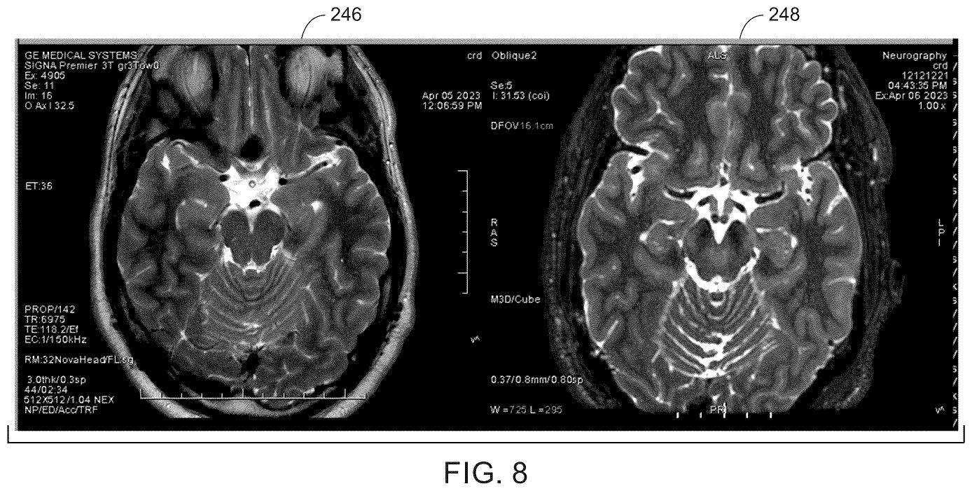

FIG. 8 is an MR image obtained of an anatomic landmark of interest (e.g., brain) of a subject (e.g., taking into account patient size), in accordance with aspects of the present disclosure;

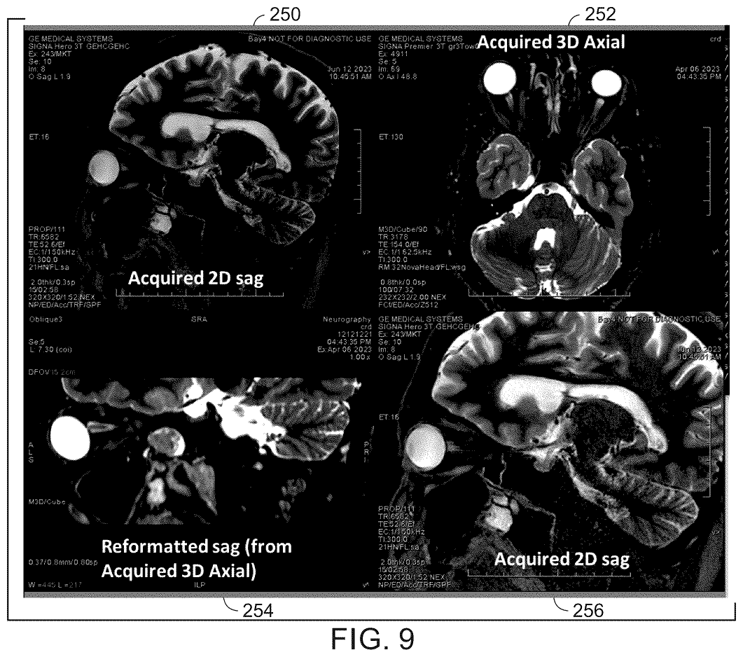

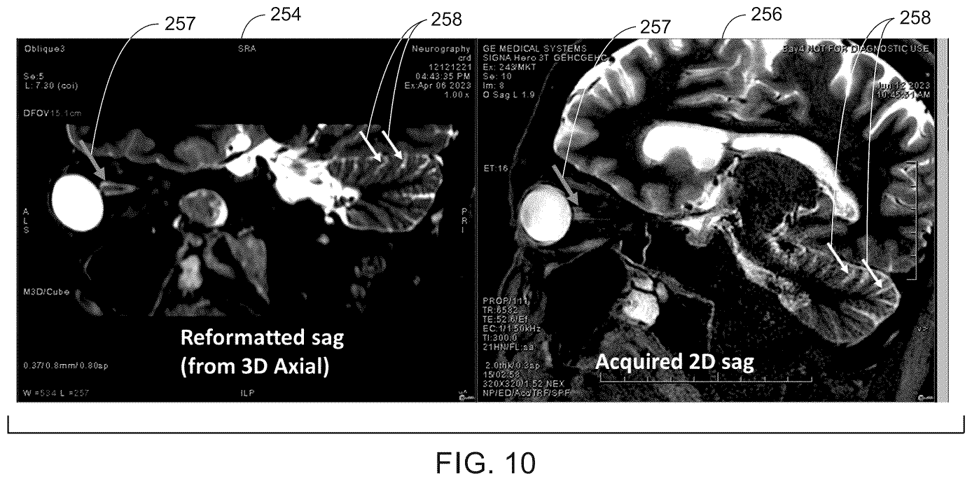

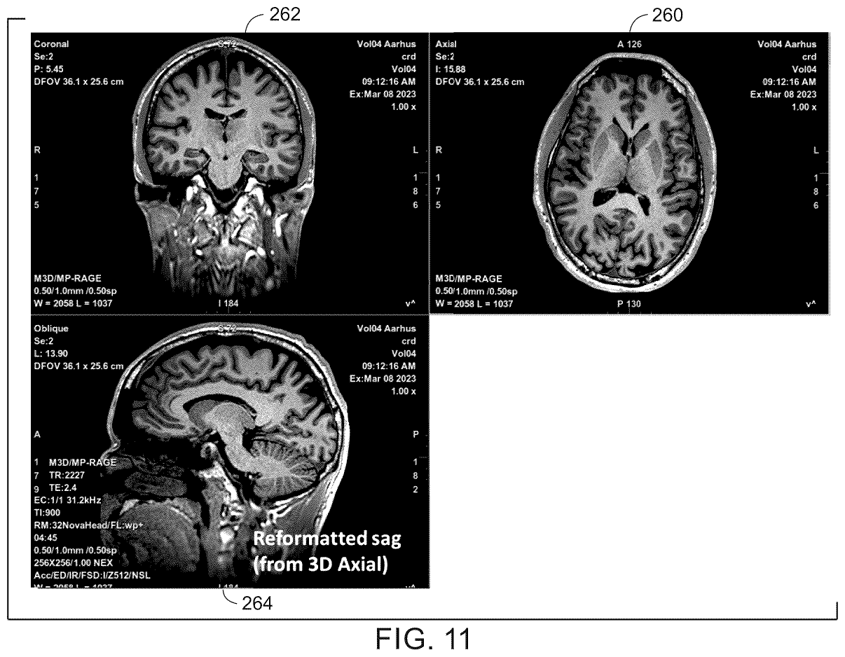

FIG. 9 depicts images for a comparison between a reformatted sagittal image and an acquired sagittal image of an anatomic landmark of interest (e.g., brain) of a subject, in accordance with aspects of the present disclosure;

FIG. 10 depicts annotated versions of the images in FIG. 9, in accordance with aspects of the present disclosure; and

FIG. 11 depicts example reformatted and sagittal images derived from a 3D acquisition, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

The term processor, processing system, or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the terms “application”, “application module” (or “module”), “engine”, or “program”, or “plugin” refers to one or more sets of computer software instructions (e.g., computer programs and/or scripts) executable by one or more processors of a computing system to provide particular functionality. Computer software instructions can be written in any suitable programming languages, such as C, C++, C#, Pascal, Fortran, Perl, MATLAB, SAS, SPSS, JavaScript, AJAX, and JAVA. Such computer software instructions can comprise an independent application with data input and data display aspects (e.g., modules). Alternatively, the disclosed computer software instructions can be classes that are instantiated as distributed objects. The disclosed computer software instructions can also be component software, for example JAVABEANS or ENTERPRISE JAVABEANS. Additionally, the disclosed applications or engines can be implemented in computer software, computer hardware, or a combination thereof.

As used herein, the terms “automatic” and “automatically” refer to actions that are performed by a computing device or computing system (e.g., of one or more computing devices) without human intervention. For example, automatically performed functions may be performed by computing devices or systems based solely on data stored on and/or received by the computing devices or systems despite the fact that no human users have prompted the computing devices or systems to perform such functions. As but one non-limiting example, the computing devices or systems may make decisions and/or initiate other functions based solely on the decisions made by the computing devices or systems, regardless of any other inputs relating to the decisions.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep-learning (DL) approaches discussed herein may be based on artificial neural networks, and may therefore encompass one or more of deep neural networks, fully connected networks, convolutional neural networks (CNNs), unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

An intelligent prescription module (e.g., trained deep learning-based algorithms or model such as AIRx™ from GE Healthcare) has enabled retrospective reformatting (IMPR) of 3D image data for multiple anatomical references of clinical interest. Such as a technique is disclosed in U.S. Publication No. 2022/0358692 filed May 4, 2021 and titled “Generating Reformatted Views of a Three-Dimensional Anatomy Scan Using Deep-Learning Estimated Scan Prescription Masks”, which is incorporated herein in its entirety for all purposes. Currently, this technique generates views of various landmarks from 3D image data available in the exam study. However, most protocols are conventionally designed for 2D acquisitions and can be repeated along different orientations by the user for various landmarks. In such scenarios, the ability to parse through protocols and suggest an appropriate 3D protocol can enhance ethe use of the capabilities of the intelligent prescription module's capabilities and save time for the user. This capability has been further improved with a deep learning-based reconstruction algorithm or model (e.g., AIR™ Recon DL or AIRDL from GE Healthcare), which reduces can time and/or increases the resolution of 3D exams.

The present disclosure provides techniques to optimal magnetic resonance (MR) exam with retrospective reformatting (IMPR) workflow that leverages integrated guidelines. In particular, a scan optimizer and recommendation system enables an optimized number of scan in a study. When a scan order is received, the system automatically refers to recommended protocols from radiology guidelines (e.g., American College of Radiology (AMR) guidelines) and/or protocols from a protocol library (e.g., having protocols from a vendor of the MRI system and/or site specific guidelines), specific to the clinical indication (for the patient or subject). These inputs refine scan suggestions and optimize acquisitions by reducing the number of rescans of each contrast over the same region of interest (ROI) along multiple views/planes. The recommendation (fine-tuned or personalized to the patient) includes performing an acquisition including a 3D acquisition (optimized with a deep learning-based reconstruction algorithm or model such as AIRDL for resolution and slice thickness) to achieve the specific contrast in the ROI at the right spatial resolution. Once the 3D acquisition is complete, the system automatically provides images (e.g., 2D images) reformatted along all planes of interest originally intended for the scan. This significantly reduces scan time, increases throughput, and avoids patient discomfort. The planes of interest are estimated from deep learning-based scan prescription masks (e.g., from an intelligent prescription model such as AIRx™), which are predicted for specific anatomical references of interest.

Scoliosis provides a first relevant clinical example for use of the disclosed techniques. For scoliosis, stacks of 2D slices are scanned along multiple planes as sagittal and coronal to provide information for axial planning. All three planes are needed to count vertebral bodies because the spine might not be aligned and visible in just one plane. Therefore, using 3D scans and then reformatting them is a common practice.

Knee joint imaging of a meniscus injury with anterior cruciate ligament tear provides a second relevant clinical example for use of the disclosed techniques. For the knee joint imaging, imaging of each of the structures is currently performed as individual 2D scans since they need specific angulation for the corresponding acquisition. This is another case where a good 3D acquisition and subsequent reformatting along the computed planes of interest would provide all necessary images from just one acquisition.

The disclosed techniques reduce overall scan time by replacing multiple 2D scans with one single 3D scan (per contrast) augmented optimally with a deep learning-based reconstruction algorithm or model such as AIRDL as per required indication. Then, the 3D scan is used to generate all required 2D reformats as per the protocol. In certain embodiments, multiple 2D scans for multiple contrasts are replaced with a single 3D scan (e.g., using a multi-delay multi-echo (MDME) scan sequence in conjunction with a synthetic MRI technique such as magnetic resonance imaging compilation (MAGiC) from GE Healthcare). The disclosed techniques provide an optimal scan time avoiding repeated scans of the same ROI (as well as the additional scan time associated with prescans). The disclosed techniques enable intelligent reformatting to all planes with respect to anatomical reference/landmark of interest needed for imaging as per the protocol and radiology guidelines (e.g., ACR guidelines).

The disclosed embodiments include a system and method for performing a scan of a subject utilizing a magnetic resonance imaging system. The system and method include receiving, via a processing system including one or more processors, at a protocol optimizer (e.g., protocol optimize module or scan optimizer and recommendation system) an electronic medical record for the subject, wherein the electronic medical record includes a scan order for scanning a region of interest of the subject for a patient specific clinical indication with a magnetic resonance scanner. The system and method also include utilizing, via the processing system, the protocol optimizer to automatically access one or more initial protocols specific to the patient specific clinical indication from radiology guidelines and/or a protocol library. The system and method further include generating, via the processing system, with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

In certain embodiments, the system and method include displaying, via the processing system, the one or more optimized protocols on a display. In certain embodiments, the system and method include performing, via the processing system, the acquisition including the 3D acquisition of the 3D image data utilizing a deep learning-based reconstruction algorithm based on the one or more optimized protocols. In certain embodiments, the system and method include automatically reformatting, via the processing system, the 3D image data to generate two-dimensional (2D) images along all planes of interest of the region of interest originally intended by the one or more initial protocols. In certain embodiments, the system and method include obtaining, via the processing system, three-plane localizer images of the subject and utilizing, via the processing system, a trained deep learning-based model to estimate a geometry plan including plane information for the automatic reformatting of the 3D image data to generate the 2D images along all the planes of interest of the region of interest. In certain embodiments, the protocol optimizer is configured to reduce the number of scans for each contrast to a single scan. In certain embodiments, the 3D image data is acquired utilizing a multi-delay multi-echo scan sequence, and the protocol optimizer is configured to reduce the number of scans for multiple contrasts to a single scan.

The disclosed embodiments also include a non-transitory computer-readable medium, the computer-readable medium including processor-executable code that when executed by a processor, causes the processor to perform actions. The actions include receiving at a protocol optimizer an electronic medical record for a subject, wherein the electronic medical record includes a scan order for scanning a region of interest of the subject for a patient specific clinical indication with a magnetic resonance scanner. The actions also include utilizing the protocol optimizer to automatically access one or more initial protocols specific to the patient specific clinical indication from radiology guidelines and/or a protocol library. The actions further include generating with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

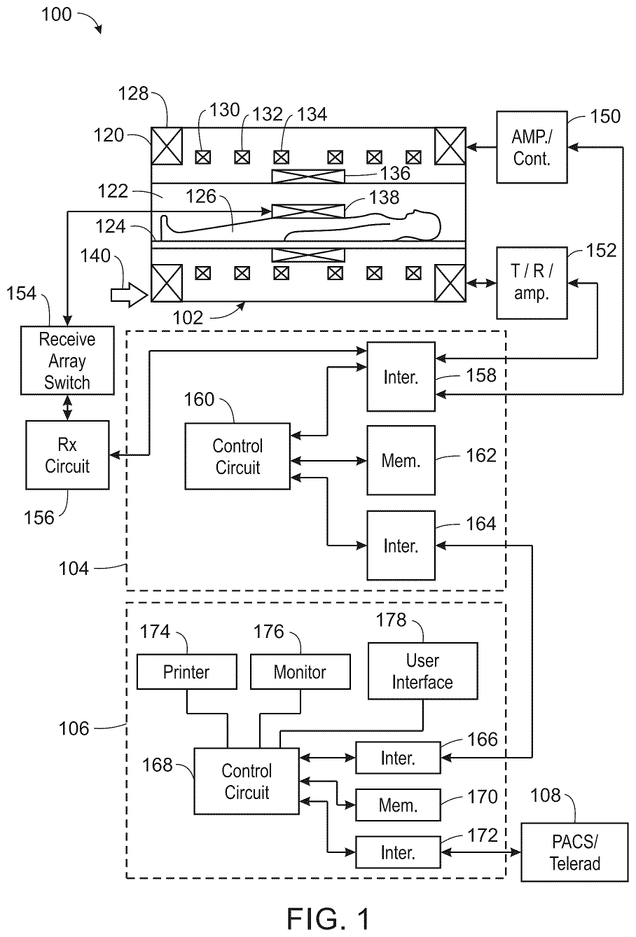

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on-or off-site. In this way, MR data may be acquired, followed by on-or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, Bo. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more neural network, algorithms, and/or modules for performing the techniques described below. For example, the memory circuit 170 may store an intelligent prescription module (e.g., trained deep learning-based algorithms or model such as AIRx™ from GE Healthcare). The memory circuit 170 may store a protocol optimizer (e.g., protocol optimize module or scan optimizer and recommendation system). The memory circuit 170 may store a deep learning-based reconstruction algorithm or model (e.g., AIR™ Recon DL or AIRDL from GE Healthcare). In certain embodiments, image reconstruction may occur on a separate computing device having processing circuitry and memory circuitry.

The programming code may enable performing a scan of a subject utilizing a magnetic resonance imaging system. The programming code may receive at a protocol optimizer (e.g., protocol optimize module or scan optimizer and recommendation system) an electronic medical record for the subject, wherein the electronic medical record includes a scan order for scanning a region of interest of the subject for a patient specific clinical indication with a magnetic resonance scanner. The programming code may utilize the protocol optimizer to automatically access one or more initial protocols specific to the patient specific clinical indication from radiology guidelines and/or a protocol library. The programming code may generate with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

In certain embodiments, the programming code may display the one or more optimized protocols on a display. In certain embodiments, the programming code may perform the acquisition including the 3D acquisition of the 3D image data utilizing a deep learning-based reconstruction algorithm based on the one or more optimized protocols. In certain embodiments, the programming code may automatically reformat the 3D image data to generate two-dimensional (2D) images along all planes of interest of the region of interest originally intended by the one or more initial protocols. In certain embodiments, the programming code may obtain three-plane localizer images of the subject and utilize a trained deep learning-based model to estimate a geometry plan including plane information for the automatic reformatting of the 3D image data to generate the 2D images along all the planes of interest of the region of interest. In certain embodiments, the protocol optimizer is configured to reduce the number of scans for each contrast to a single scan. In certain embodiments, the 3D image data is acquired utilizing a multi-delay multi-echo scan sequence, and the protocol optimizer is configured to reduce the number of scans for multiple contrasts to a single scan.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

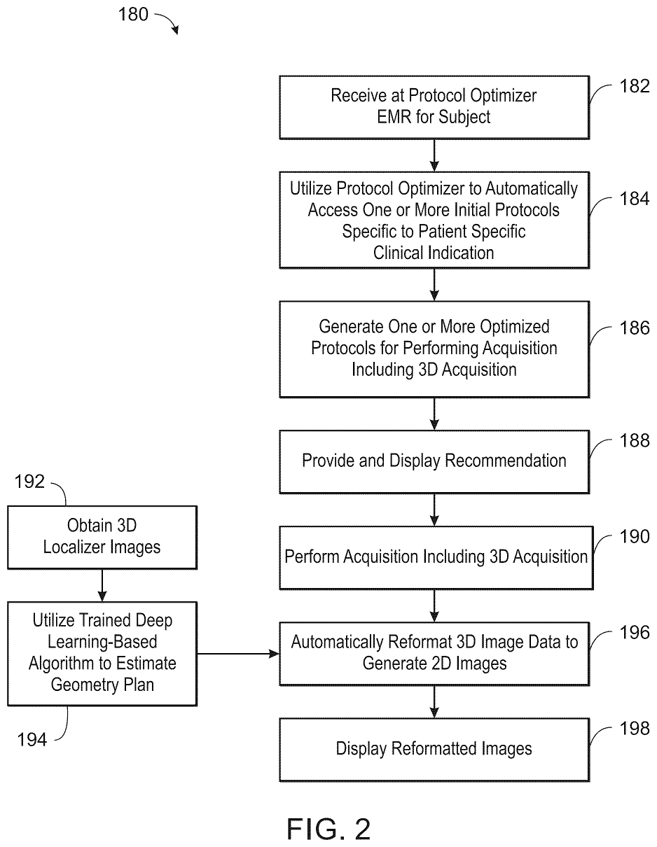

FIG. 2 illustrates a flow diagram of a method 180 for performing a scan of a patient utilizing the MRI system 100 in FIG. 1 utilizing an improved MR scanning workflow. One or more steps of the method 180 may be performed by processing circuitry of the magnetic resonance imaging system 100 in FIG. 1. One or more of the steps of the method 180 may be performed simultaneously or in a different order from the order depicted in FIG. 2.

The method 180 includes receiving at a protocol optimizer an electronic medical record (EMR) for the subject (block 182). The electronic medical record includes a scan order for scanning a region of interest of the subject for a patient specific clinical indication (e.g., unique medical reason or set of symptoms for the scan that takes into account individual medical history, current condition, and other relevant factors (lab results, genetic information, etc.) with a magnetic resonance scanner (e.g., scanner 102 in FIG. 1). The scan order includes the anatomical structure(s) and/or region(s) of interest to be scanned and any special instructions (e.g., if a contrast is to utilized, presence of metal implant, etc.).

The method 180 also includes utilizing the protocol optimizer to automatically access one or more initial protocols specific to the patient specific clinical indication from radiology guidelines and/or a protocol library (block 184). The radiology guidelines may be ACR guidelines and/or guidelines from another institution. The protocol library may include protocols from a vendor of the MRI system and/or site specific guidelines.

The method 180 further includes generating with the protocol optimizer one or more optimized protocols (as part of a recommendation) from the one or more initial protocols for performing a three-dimensional (3D) acquisition of 3D image data of the region of interest of the subject (block 186). The acquisition may include more than one 3D acquisition. In certain embodiments, the acquisition may include one or more 2D acquisitions. The protocol optimizer also provides an optimal total scan time (e.g., on the MR scanner). The protocol optimizer is configured to reduce a number of scans for each contrast that is required by the patient specific clinical indication over the region of interest along multiple planes. In certain embodiments, the protocol optimizer is configured to reduce the number of scans for each contrast to a single scan. In certain embodiments, the 3D image data is acquired utilizing a multi-delay multi-echo (MDME) scan sequence, and the protocol optimizer is configured to reduce the number of scans for multiple contrasts to a single scan. For example, the MDME scan sequence may be utilized in conjunction with a synthetic MRI technique such as magnetic resonance imaging compilation (MAGiC) from GE Healthcare). For example, MAGiC provides post-processing pipeline for an MRI scan that generates multiple contrast-weighted images from a single scan. For example, MAGiC can generate T1, T2, short tau inversion recovery (STIR), T1 fluid-attenuate inversion recovery (FLAIR), T2 FLAIR, and proton density (PD) contrast images from a single scan.

The method 180 even further includes providing and displaying a recommendation on a display (e.g., on a graphical user interface such as on an operator console of the MRI system) (block 188). The recommendation may include an initial protocol(s) originally intended for the scan. The recommendation includes the optimized protocol(s) for the scan generate by the protocol optimizer (which includes a recommended 3D acquisition). The recommended 3D scans have the required spatial resolution and anatomical ROI as per the guideline and the landmarks needed for the patient-specific clinical indication. The recommended 3D scans provide a good visualization of the ROI.

The method 180 still further includes performing the acquisition including the 3D acquisition of the 3D image data based on the one or more optimized protocols (block 190). The 3D acquisition may include one or more 3D scans. In certain embodiments, the 3D acquisition includes utilizing (for optimization of resolution and slice thickness) a deep learning-based reconstruction algorithm or model such as AIR™ Recon DL or AIRDL from GE Healthcare, which reduces can time and/or increases the resolution of 3D exams. AIRDL may perform denoising, de-streaking, and super-resolving. AIRDL utilizes raw complex k-space data as an input in reconstructing MR images. In certain embodiments, the 3D acquisition may utilize an MDME scan sequence in conjunction with a synthetic MRI technique such as MAGiC.

The method 180 further includes obtaining three-plane localizer images of the subject (e.g., of the region of interest of the subject) (block 192). The three-plane localizer images may be acquired as part of carrying out the optimized protocol on the subject with the MR scanner (e.g., as a scan prior to the 3D acquisition). The method 180 even further includes utilizing a trained deep learning-based algorithm or model to estimate a geometry plan including plane information for automatic reformatting of the 3D image data to generate 2D images along all the planes of interest of the region of interest (block 194). In certain embodiments, the trained deep learning-based algorithm or model is an intelligent prescription module (such as AIRₓ™ from General Electric Healthcare) that is configured to automatically detect anatomic landmark of interest in the three-plane localizer images of the subject and determine a geometry plan (e.g., prescribed slices including center and orientation) of the scan of the anatomic landmark of interest (region of interest) including extents of the anatomic landmark of interest of the subject based on the three-plane localizer images. In particular, the intelligent prescription module utilizes predicted prescription masks for the specific anatomical references of interest (region of interest). The method 180 still further includes automatically reformatting the 3D image data (e.g., utilizing the geometry plan) to generate 2D images (focused to region of interest with a small field of view (FOV)) along all planes of interest of the region of interest originally intended by the one or more initial protocols (block 196).

The method 180 further includes displaying the reformatted images (i.e., 2D images) on the graphical user interface on a display and/or saving the reformatted images (block 197). In certain embodiments, the method 180 enables user selection or configuration save digital imaging and communications in medicine (DICOM) images.

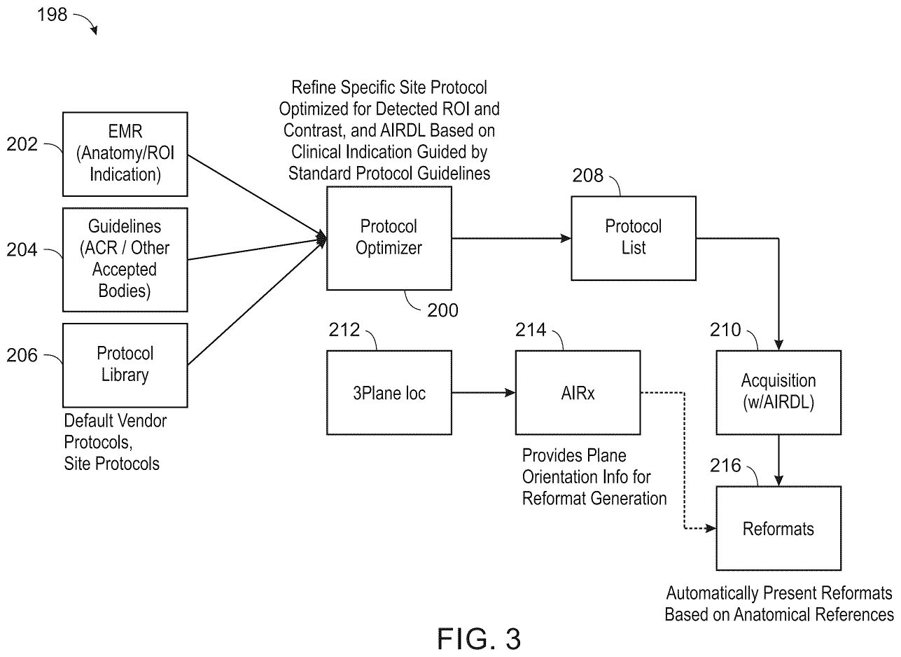

FIG. 3 illustrates a schematic diagram of a workflow 198 (e.g., optimized workflow) for performing a scan of a patient. The workflow 198 includes utilizing receiving at a protocol optimizer 200 an electronic medical record 202 for the subject. The electronic medical record 202 includes a scan order for scanning a region of interest of the subject for a patient specific clinical indication (e.g., unique medical reason or set of symptoms for the scan that takes into account individual medical history, current condition, and other relevant factors (lab results, genetic information, etc.) with a magnetic resonance scanner (e.g., scanner 102 in FIG. 1). The scan order includes the anatomical structure(s) and/or region(s) of interest to be scanned and any special instructions (e.g., if a contrast is to utilized, presence of metal implant, etc.).

The workflow 198 also includes utilizing the protocol optimizer 200 to automatically access one or more initial protocols specific to the patient specific clinical indication from radiology guidelines 204 and/or a protocol library 206. The radiology guidelines 204 may be ACR guidelines and/or guidelines from another institution. The protocol library 206 may include protocols from a vendor of the MRI system and/or site specific guidelines.

The workflow 198 further includes generating with the protocol optimizer 200 a protocol list 208. The protocol lists 208 includes one or more optimized protocols (as part of a recommendation) from the one or more initial protocols for performing a three-dimensional (3D) acquisition of 3D image data of the region of interest of the subject. The protocol optimizer 200 also provides an optimal total scan time (e.g., on the MR scanner). The protocol optimizer 200 is configured to reduce a number of scans for each contrast that is required by the patient specific clinical indication over the region of interest along multiple planes. In certain embodiments, the protocol optimizer 200 is configured to reduce the number of scans for each contrast to a single scan. In certain embodiments, the 3D image data is acquired utilizing an MDME scan sequence, and the protocol optimizer 200 is configured to reduce the number of scans for multiple contrasts to a single scan. For example, the MDME scan sequence may be utilized in conjunction with a synthetic MRI technique such as MAGiC from GE Healthcare.

The workflow 198 still further includes performing an acquisition including the 3D acquisition of the 3D image data based on the one or more optimized protocols as indicated by reference numeral 210. The 3D acquisition may include one or more 3D scans. In certain embodiments, the 3D acquisition includes utilizing (for optimization of resolution and slice thickness) a deep learning-based reconstruction algorithm or model such as AIR™ Recon DL or AIRDL from GE Healthcare, which reduces can time and/or increases the resolution of 3D exams. AIRDL may perform denoising, de-streaking, and super-resolving. AIRDL utilizes raw complex k-space data as an input in reconstructing MR images. In certain embodiments, the 3D acquisition may utilize an MDME scan sequence in conjunction with a synthetic MRI technique such as MAGiC. In certain embodiments, the acquisition may include one or more 2D acquisitions.

The workflow 198 further includes obtaining three-plane localizer images of the subject (e.g., of the region of interest of the subject) as indicated by reference numeral 212. The three-plane localizer images may be acquired as part of carrying out the optimized protocol on the subject with the MR scanner (e.g., as a scan prior to the 3D acquisition). The workflow 198 even further includes utilizing a trained deep learning-based algorithm or model to estimate a geometry plan including plane information for automatic reformatting of the 3D image data to generate 2D images along all the planes of interest of the region of interest as indicated by reference numeral 214. In certain embodiments, the trained deep learning-based algorithm or model is an intelligent prescription module (such as AIRₓ™ from General Electric Healthcare) that is configured to automatically detect anatomic landmark of interest in the three-plane localizer images of the subject and determine a geometry plan (e.g., prescribed slices including center and orientation) of the scan of the anatomic landmark of interest (region of interest) including extents of the anatomic landmark of interest of the subject based on the three-plane localizer images. In particular, the intelligent prescription module utilizes predicted prescription masks for the specific anatomical references of interest (region of interest). The workflow 198 still further includes automatically reformatting the 3D image data (e.g., utilizing the geometry plan) to generate 2D images (focused to region of interest with a small FOV) along all planes of interest of the region of interest originally intended by the one or more initial protocols as indicated by reference numeral 216.

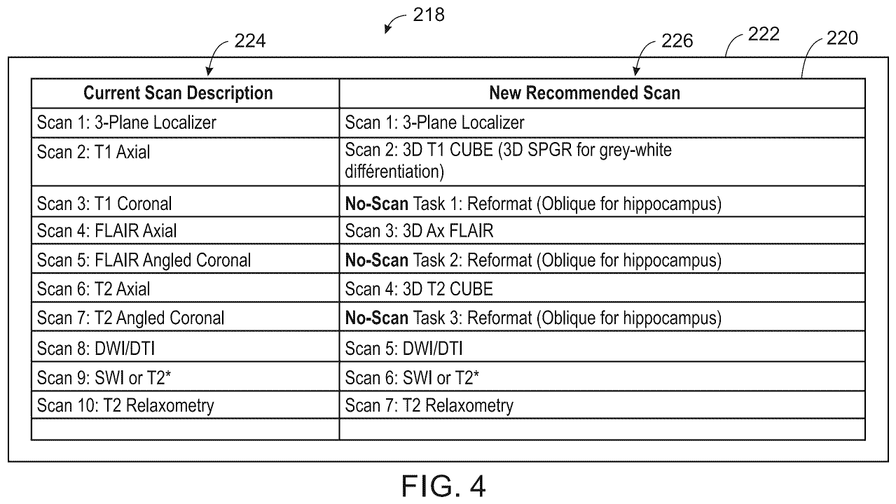

FIG. 4 depicts an example recommendation 218 provided on a graphical user interface 220 of a display 222 for a temporal lobe epilepsy protocol. High resolution scans are needed to detect malformations of cortical development and sclerosis. As depicted, the recommendation 218 includes the current scan description 224 (initial scan description or initial protocol) having the current scan steps. As depicted, the recommendation 218 includes a new recommended scan description 226 (optimized scan description or optimized protocol) personalized (i.e., fine-tuned) to the subject. As depicted, the new recommended scan description 226 recommends a single 3D scan for each contrast that requires different views/planes and then utilizing automatically generated reformats of planes to meet user expectations per site protocols and radiology guidelines. As one example, instead of performing both a T1 axial scan and a T1 coronal scan, a single 3D CUBE scan (including a 3D spoiled gradient-recalled echo (SPGR) scan for grey-white differentiation) is recommended along with automatically generating a reformatted view that is oblique for the hippocampus. The new recommended scan description 226 reduces the number of scans from ten to seven (which includes three 3D scans) without a reduction in diagnostic confidence. The new recommended scan description 226 optimizes the total scan time as well. In certain embodiments, the number of scans may be reduced even further utilizing an MDME scan sequence in conjunction with MAGiC, which can generate different contrast images from a single 3D scan.

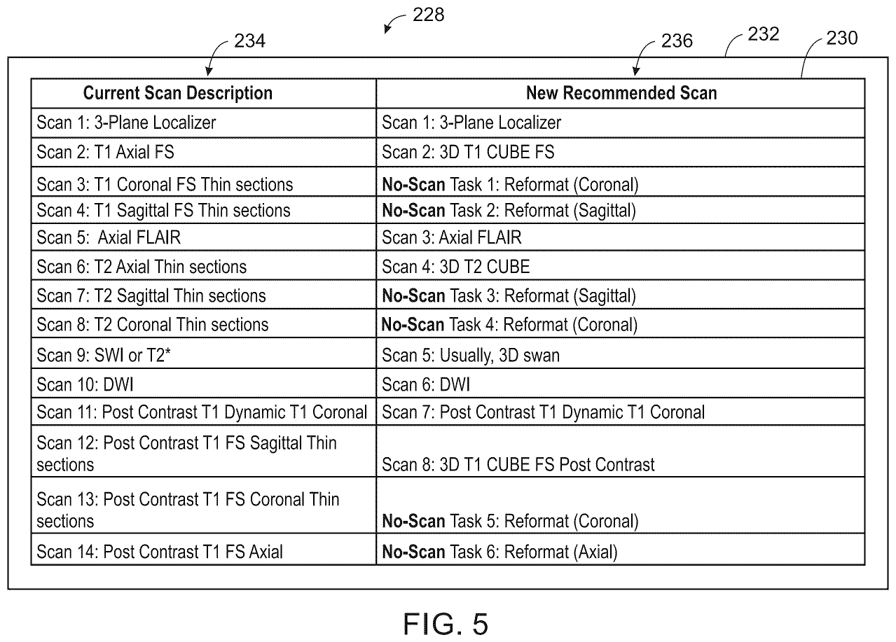

FIG. 5 depicts an example recommendation 228 provided on a graphical user interface 230 of a display 232 for a complete pituitary protocol. High resolution scans are needed for pituitary micro adenoma cases. As depicted, the recommendation 228 includes the current scan description 234 (initial scan description or initial protocol) having the current scan steps. As depicted, the recommendation 228 includes a new recommended scan description 236 (optimized scan description or optimized protocol) personalized (i.e., fine-tuned) to the subject. As depicted, the new recommended scan description 236 recommends a single 3D scan for each contrast that requires different views/planes and then utilizing automatically generated reformats of planes to meet user expectations per site protocols and radiology guidelines. As one example, instead of performing a T1 axial fat saturation (FS) scan, a T1 coronal FS thin sections scan, and a T1 sagittal FS thin section scan, a single 3D CUBE FS scan is recommended along with automatically generating reformatted coronal and sagittal views. The new recommended scan description 236 reduces the number of scans from fourteen to eight (which includes six 3D scans) without a reduction in diagnostic confidence. The new recommended scan description 236 optimizes the total scan time as well. In certain embodiments, the number of scans may be reduced even further utilizing an MDME scan sequence in conjunction with MAGiC, which can generate different contrast images from a single 3D scan.

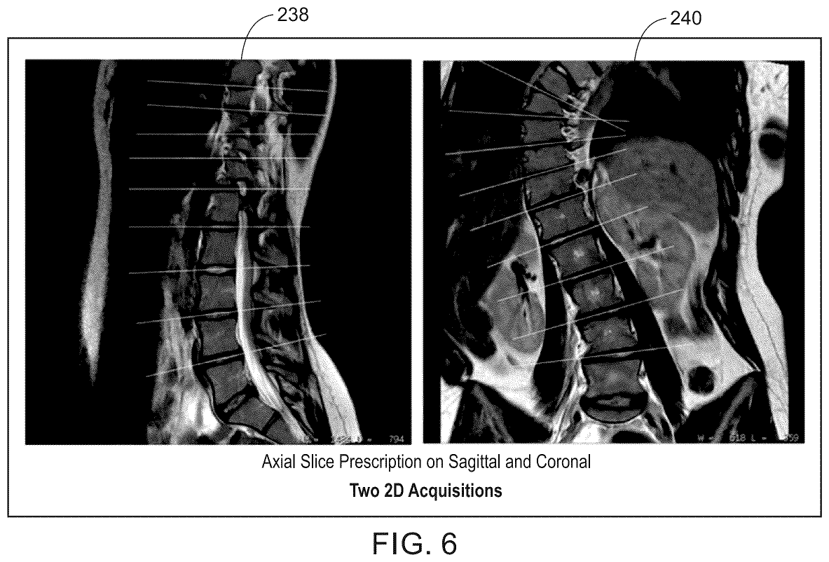

In the scoliosis cases, for axial slice planning both sagittal and coronal images are required to count the vertebrae and to update slice orientations for appropriate angulations. FIG. 6 depicts a sagittal image 238 and a coronal image 240 acquired with two 2D acquisitions (with an MR scanner) for an axial slice prescription. FIG. 7 depicts a thoraco-lumbar spine sagittal T2 image 242 and a thoraco-lumbar spine coronal T2 image 244 (i.e., sagittal and coronal reformats) derived from a single 3D acquisition utilizing the method 180 in FIG. 2 and the workflow 198 in FIG. 3. Images from a single 3D acquisition are reformatted to obtain the relevant sagittal and coronal views (i.e., images 242, 244) to get the optimal axial orientation prescriptions.

FIG. 8 depicts an image 246 of a brain of a subject derived from a 2D acquisition and a reformatted image 248 of the brain from a 3D acquisition. The image 246 is a Periodically Rotated Overlapping Parallel Lines with Enhanced Reconstruction (PROPELLER) axial (no fat-saturation) image (in particular, axial section through mid-brain area). The reformatted image 248 is a 3D CUBE image (with fat saturation) reformatted to a mid-brain axial section derived from the 3D acquisition utilizing the method 180 in FIG. 2 and the workflow 198 in FIG. 3. The original data was acquired on a Microstructure Anatomy Gradient for Neuroimaging with Ultrafast Scanning (MAGNUS) 3T scanner. The information (i.e. cerebellar folio (fine folds in the cerebellum) as well as the grey matter and white matter contrast) is preserved and better represented in the reformatted image 248.

FIG. 9 depicts an sagittal image 250 of a brain of a subject derived from a 2D acquisition, an axial image 252 of the brain derived from a 3D acquisition, a reformatted sagittal image 254 (e.g., 3D CUBE reformatted) of the brain from the 3D acquisition, and an image 256 (which is zoomed portion of the axial image 250). The image 246 is a Periodically Rotated Overlapping Parallel Lines with Enhanced Reconstruction (PROPELLER) axial (no fat-saturation) image (in particular, axial section through mid-brain area). The reformatted sagittal image 254 was derived utilizing the method 180 in FIG. 2 and the workflow 198 in FIG. 3. The original data was acquired on a Microstructure Anatomy Gradient for Neuroimaging with Ultrafast Scanning (MAGNUS) 3Tscanner. FIG. 10 depicts annotations of the axial image 250 and the reformatted sagittal image 254. In both images 250, 254, the visibility of the optic nerve is preserved. The optic nerve is indicated by arrow 257. The cerebellar folia (fine folds in the cerebellum) are frequently used for evaluation because they are among the thinnest structures. This makes them ideal for assessing reformat performance, particularly in terms of blurriness and loss of contrast. In both images 250, 254, the posterior cerebellar folia (indicated by arrows 258) is preserved. In both images 250, 254, T2W contrast is preserved.

FIG. 11 depicts examples of reformatted and sagittal images derived from a 3D acquisition. Image 260 is an axial image of a brain of a subject derived from a 3D acquisition. Image 262 is a reformatted coronal image of the brain derived from the 3D axial acquisition. Image 264 is a reformatted sagittal image of the brain derived from the 3D axial acquisition. The images 262, 264 were derived utilizing the method 180 in FIG. 2 and the workflow 198 in FIG. 3. This demonstrates that reformatting along other planes from an acquired 3D acquisition does not degrade image quality.

Technical effects of the disclosed subject matter include providing an optimal magnetic resonance (MR) exam with retrospective reformatting (IMPR) workflow that leverages integrated guidelines. In particular, an automated solution is provided that optimizes scans by recommending 3D scans further leveraging deep learning-based reconstruction algorithm capabilities) and automatically generating reformats of planes per radiology guidelines and patient-specific indications. Technical effects of the disclosed subject matter include reducing scan time. Technical effects of the disclosed subject matter include increasing throughput. Technical effects of the disclosed subject matter include avoiding patient discomfort.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for performing a scan of a subject utilizing a magnetic resonance imaging system, comprising:
receiving, via a processing system comprising one or more processors, at a protocol optimizer an electronic medical record for the subject, wherein the electronic medical record comprises a scan order for scanning a region of interest of the subject for a patient specific clinical indication with a magnetic resonance scanner;
utilizing, via the processing system, the protocol optimizer to automatically access one or more initial protocols specific to the patient specific clinical indication from radiology guidelines and/or a protocol library; and
generating, via the processing system, with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

2. The computer-implemented method of claim 1, further comprising displaying, via the processing system, the one or more optimized protocols on a display.

3. The computer-implemented method of claim 1, further comprising performing, via the processing system, the acquisition including the 3D acquisition of the 3D image data utilizing a deep learning-based reconstruction algorithm based on the one or more optimized protocols.

4. The computer-implemented method of claim 3, further comprising automatically reformatting, via the processing system, the 3D image data to generate two-dimensional (2D) images along all planes of interest of the region of interest originally intended by the one or more initial protocols.

5. The computer-implemented method of claim 4, further comprising:
obtaining, via the processing system, three-plane localizer images of the subject; and
utilizing, via the processing system, a trained deep learning-based model to estimate a geometry plan including plane information for the automatic reformatting of the 3D image data to generate the 2D images along all the planes of interest of the region of interest.

6. The computer-implemented method of claim 1, wherein the protocol optimizer is configured to reduce the number of scans for each contrast to a single scan.

7. The computer-implemented method of claim 1, wherein the 3D image data is acquired utilizing a multi-delay multi-echo scan sequence, and the protocol optimizer is configured to reduce the number of scans for multiple contrasts to a single scan.

8. A system for performing a scan of a subject utilizing a magnetic resonance imaging system, comprising:
a memory encoding processor-executable routines; and
a processing system comprising one or more processors and configured to access the memory and to execute the processor-executable routines, wherein the processor-executable routines, when executed by the processing system, cause the processing system to:
receive at a protocol optimizer an electronic medical record for the subject, wherein the electronic medical record comprises a scan order for scanning a region of interest of the subject for a patient specific clinical indication with a magnetic resonance scanner;
utilize the protocol optimizer to automatically access one or more initial protocols specific to the patient specific clinical indication from radiology guidelines and/or a protocol library; and
generate with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

9. The system of claim 8, wherein the processor-executable routines, when executed by the processing system, further cause the processing system to display the one or more optimized protocols on a display.

10. The system of claim 8, wherein the processor-executable routines, when executed by the processing system, further cause the processing system to perform the acquisition including the 3D acquisition of the 3D image data utilizing a deep learning-based reconstruction algorithm based on the one or more optimized protocols.

11. The system of claim 10, wherein the processor-executable routines, when executed by the processing system, further cause the processing system to automatically reformat the 3D image data to generate two-dimensional (2D) images along all planes of interest of the region of interest originally intended by the one or more initial protocols.

12. The system of claim 11, wherein the processor-executable routines, when executed by the processing system, further cause the processing system to:
obtain three-plane localizer images of the subject; and
utilize a trained deep learning-based model to estimate a geometry plan including plane information for the automatic reformatting of the 3D image data to generate the 2D images along all the planes of interest of the region of interest.

13. The system of claim 8, wherein the protocol optimizer is configured to reduce the number of scans for each contrast to a single scan.

14. The system of claim 8, wherein the 3D image data is acquired utilizing a multi-delay multi-echo scan sequence, and the protocol optimizer is configured to reduce the number of scans for multiple contrasts to a single scan.

15. A non-transitory computer-readable medium, the non-transitory computer-readable medium comprising processor-executable code that when executed by a processing system comprising one or more processors, causes the processing system to:
receive at a protocol optimizer an electronic medical record for a subject, wherein the electronic medical record comprises a scan order for scanning a region of interest of the subject for a patient specific clinical indication with a magnetic resonance scanner;
utilize the protocol optimizer to automatically access one or more initial protocols specific to the patient specific clinical indication from radiology guidelines and/or a protocol library; and
generate with the protocol optimizer one or more optimized protocols from the one or more initial protocols for performing an acquisition including a three-dimensional (3D) acquisition of 3D image data of the region of interest of the subject, wherein the protocol optimizer is configured to reduce a number of scans for each contrast that is required by the patient specific clinical indication over the region of interest along multiple planes.

16. The non-transitory computer-readable medium of claim 15, wherein the processor-executable code, when executed by the processing system, further cause the processing system to display the one or more optimized protocols on a display.

17. The non-transitory computer-readable medium of claim 15, wherein the processor-executable code, when executed by the processing system, further cause the processing system to:
perform the acquisition including the 3D acquisition of the 3D image data utilizing a deep learning-based reconstruction algorithm based on the one or more optimized protocols; and
automatically reformat the 3D image data to generate two-dimensional (2D) images along all planes of interest of the region of interest originally intended by the one or more initial protocols.

18. The non-transitory computer-readable medium of claim 17, wherein the processor-executable code, when executed by the processing system, further cause the processing system to:
obtain three-plane localizer images of the subject; and
utilize a trained deep learning-based model to estimate a geometry plan including plane information for the automatic reformatting of the 3D image data to generate the 2D images along all the planes of interest of the region of interest.

19. The non-transitory computer-readable medium of claim 15, wherein the protocol optimizer is configured to reduce the number of scans for each contrast to a single scan.

20. The non-transitory computer-readable medium of claim 15, wherein the 3D image data is acquired utilizing a multi-delay multi-echo scan sequence, and the protocol optimizer is configured to reduce the number of scans for multiple contrasts to a single scan.
